(19) United States
(12) Patent Application Publication
(10) Pub. No.: US 2024/0156857 A1
(43) Pub. Date: May 16, 2024

(54) HYDRATION FORMULATION

(71) Applicant: Bausch + Lomb Ireland Limited, Dublin (IE)

(72) Inventors: Martin J. COFFEY, Pittsford, NY (US); Zora Marlowe, Rochester, NY (US)

(21) Appl. No.: 18/506,909

(22) Filed: Nov. 10, 2023

Related U.S. Application Data
(60) Provisional application No. 63/383,435, filed on Nov. 11, 2022.

(51) Int. Cl. A61K 31/728; A61K 31/047; A61K 31/198; A61K 31/375; A61K 31/525; A61K 31/7016; A61K 47/02; A61K 47/12; A61P 27/02

(52) U.S. Cl. CPC A61K 31/728; A61K 31/047; A61K 31/198; A61K 31/375; A61K 31/525; A61K 31/7016; A61K 47/02; A61K 47/12; A61P 27/02

(57) ABSTRACT

The present disclosure describes formulations and methods for hydrating dry eyes. The formulations comprise at least one natural humectant, at least one amino acid, and at least one vitamin, wherein the formulation is preservative free, and may provide effective hydration, lubrication, and/or nourishment of the eyes.

The present disclosure is related to formulations comprising at least one natural humectant, at least one amino acid, and at least one vitamin, wherein the formulation is preservative free. The present disclosure also provides methods of treating, improving, and/or ameliorating dryness; removing irritants and/or debris; maintaining eye hygiene; and/or treating, improving, or ameliorating dry eye and symptoms thereof. In some embodiments, the formulations and methods provide for cleansing, cleaning, conditioning, moisturizing, lubricating, hydrating, nourishing, soothing, calming, and/or refreshing ocular surface tissues.

The formulations of the present disclosure may rebuild and/or nurture the homeostasis of the ocular surface and may provide essential nutrients to a compromised tear fluid to restore tear film integrity.

Dry eye is defined by the DEWS Definition and Classification Subcommittee as a multifactorial disease of the tears and ocular surface that results in symptoms of discomfort, visual disturbance, and tear instability with potential damage to the ocular surface, accompanied by increased osmolarity of the tear film and inflammation of the ocular surface. (Ubels et al., Curr. Eye Res., 28:437-444, 2002). Dry eye is a disorder of the ocular surface due to tear deficiency, excessive tear evaporation, or incorrect composition of tears. For most patients with mild dry eye symptoms, over-the-counter dry eye drops (OTC-DED) are the first line of therapy. These products offer temporary relief by providing replacement of naturally produced tears in patients with aqueous tear deficiency, acting as lubricants, diluting pro-inflammatory substances and/or protecting against osmotic stress. Some drops may contain lipids to restore the lipid layer of the tear film.

A recent study has shown a link between sleep deprivation and dry eye syndrome. Amino acids such as arginine, leucine, and taurine were found to be lower in the tears in a sleep derived model. Leucine is the main component of proteoglycans found in the corneal stroma and aids in corneal healing. Arginine is necessary for the expression of beta-defensins, which are part of the immune system. Taurine is a transporter necessary for osmoregulatory action. The formulation of the present disclosure assists in replenishing amino acids in tears.

### BRIEF DESCRIPTION OF FIGURES

In order to provide an understanding of the embodiments of the present disclosure, reference is made to the appended figures. The figures are exemplary and should not be construed as limiting the disclosure.

FIG. 1 is a graph showing metabolic activity of a transformed human corneal epithelial cell line (ATCC-HCEC) cultured in EpiLife medium until confluent and after 24 hour exposure to 75% EpiLife: 25% HBSS or dry eye drop products and solution adjusted to 400 and 450 mOsm/kg with NaCl (n=3 for all tests); mean IL-6 response and SEM from aqueous and emulsion-based formulations; and two-way ANOVA with Dunnett's multiple comparisons showed a significantly lower (*p<0.05) or higher (**p<0.05) difference in mean metabolic activity versus HBSS control.

FIG. 2 is a graph showing cytokine IL-6 response of ATCC-HCEC cultured in EpiLife medium until confluent and after 24 hour exposure to 75% EpiLife: 25% HBSS or dry eye drop products and solution adjusted to 400 and 450 mOsm/kg with NaCl (n=3 for all tests); mean metabolic activity from aqueous and emulsion based formulations are shown; and two-way ANOVA with Dunnett's multiple comparisons showed a significantly higher (*p<0.05) or lower (**p<0.05) difference in mean IL-6 response versus HBSS control.

FIG. 3 is a graph showing chemokine IL-8 response of ATCC-HCEC cultured in EpiLife medium until confluent and after 24 hour exposure to 75% EpiLife: 25% HBSS or dry eye drop products and solution adjusted to 400 and 450 mOsm/kg with NaCl (n=3 for all tests); mean metabolic activity from aqueous and emulsion based formulations are shown; and two-way ANOVA with Dunnett's multiple comparisons showed a significantly higher (*p<0.05) or lower (**p<0.05) difference in IL-8 response versus HBSS control.

FIG. 4(A-F) are graphs showing Metabolic Activity of Riken-HCEC after 24 hour exposure to 75% DMEM/F12: 25% HBSS, OTC vitamin drop or formulation components and solutions adjusted to 400, 450 and 500 mOsm/kg with NaCl (n=3 for all tests). Mean metabolic activity and SEM from HBSS control and formulations are shown; and two-way ANOVA with Dunnett's multiple comparisons showed a significantly higher (*p<0.05) or lower (**p<0.05) difference in mean metabolic activity versus HBSS control, FIGS. 4A and D; versus Full Formulation minus one component, FIGS. 4 B and E; and versus test formulations, FIGS. 4C and F. Comparisons of the same test formulation versus the test formulation 315 mOsm/kg control are in FIGS. 4A, B and C. Comparisons of the test formulations at the same osmolarity versus the HBSS control (FIG. 4D); versus full formulations (FIG. 4E); versus test formulations (FIG. 4F).

FIG. 5(A-F) are graphs showing IL-6 response of Riken-HCEC after 24 hour exposure to 75% DMEM/F12: 25% HBSS, OTC vitamin drop or formulation components and solutions adjusted to 400, 450 and 500 mOsm/kg with NaCl (n=3 for all tests). Mean IL-6 responses and SEM from HBSS control and formulations are shown in FIGS. 5A-F. Two-way ANOVA with Dunnett's multiple comparisons showed a significantly higher (*p<0.05) or lower (**p<0.05) difference in mean IL-6 versus HBSS control, FIGS. 5A and D; versus full formulation minus one component, FIGS. 5B and E; and versus test formulations, FIGS. 5C and F. Comparisons of the same test formulation versus the test formulation 315 mOsm/kg control are in FIGS. 5A, B, and C. Comparisons of the test formulations at the same osmolarity versus the HBSS control (FIG. 5D); versus full formulations (FIG. 5E); versus test formulations (FIG. 5F).

FIG. 6(A-F) are graphs showing IL-8 response of Riken-HCEC after 24 hour exposure to 75% DMEM/F12: 25% HBSS. OTC vitamin drop or formulation components and solutions adjusted to 400, 450 and 500 mOsm/kg with NaCl (n=3 for all tests). Mean IL-8 responses and SEM from HBSS control and formulations are shown in FIGS. 6A-F. Two-way ANOVA with Dunnett's multiple comparisons showed a significantly higher (*p<0.05) or lower (**p<0.05) difference in mean IL-8 versus HBSS control, FIGS. 6A and D; versus full formulation minus one component, FIGS. 6B and E; and versus test formulations, FIGS. 6C and F. Comparisons of the same test formulation versus the test formulation 315 mOsm/kg control are in FIGS. 6A, B, and C. Comparisons of the test formulations at the same osmolarity versus the HBSS control (FIG. 6D); versus full formulations (FIG. 6E); versus test formulations (FIG. 6F).

FIG. 7(A-F) are graphs showing MCP-1 response of Riken-HCEC after 24 hour exposure to 75% DMEM/F12: 25% HBSS, OTC vitamin drop or formulation components and solutions adjusted to 400, 450 and 500 mOsm/kg with NaCl (n=3 for all tests). Mean MCP-1 responses and SEM from HBSS control and formulations are shown in FIGS. 7A-F. Two-way ANOVA with Dunnett's multiple comparisons showed a significantly higher (*p<0.05) or lower (**p<0.05) difference in mean MCP-1 versus HBSS control, FIGS. 7A and D; versus full formulation minus one component, FIGS. 7B and E; and versus test formulations, FIGS. 7C and F. Comparisons of the same test formulation versus the test formulation 315 mOsm/kg control are in FIGS. 7A, B, and C. Comparisons of the test formulations at the same osmolarity versus the HBSS control (FIG. 7D); versus full formulations (FIG. 7E); versus test formulations (FIG. 7F).

The present disclosure will now be described more fully. However, the disclosure may be embodied in many different forms and should not be construed as limited to the embodiments set forth herein.

Definitions of certain terms as used in this application are provided below. Unless defined otherwise, all technical and scientific terms used herein have the normal and common meaning that would be commonly understood by one of ordinary skill in the art to which this disclosure belongs.

As used herein, “a,” “an,” and “the” refer to one or more (i.e., to at least one) of the grammatical object of the article.

As used herein, the terms “hydrating,” “lubricating,” and “nourishing” are used interchangeably and all refer to reduction of dryness, prevention of water loss, tear evaporation protection, restoration of natural moisture balance of the tear film, or any combination of the foregoing.

The terms “patient,” “subject,” “individual,” and the like, as used herein, are interchangeable and all refer to any animal, which may be a human or a non-human animal.

As will be understood by one of ordinary skill in the art, when disclosed herein, each range includes all possible subranges as well as individual numerical values within that range. For example, a range of “1.0 to 5.0” includes and would be understood to specifically disclose subranges such as “1.0 to 3.0,” “1.5 to 3.7,” “2.1 to 4.3,” etc., as well as all individual numbers within the disclosed range, for example, 1.0, 1.1, 1.2, 1.3, etc.

As used herein, a formulation is “stable” if there is no visible appearance of flocculation and/or phase separation.

As used herein, a formulation is “shelf stable” if, after being stored at 20-25° C. for 24 months, there is no visible appearance of flocculation and/or phase separation.

As used herein, a formulation is “free of” a compound when said compound is not added during manufacture of the formulation.

As noted above, the present disclosure is related to formulations for hydrating eyes comprising at least one natural humectant, at least one amino acid, and at least one vitamin, wherein the formulation is preservative free. The formulations may be useful for treating, improving, and/or ameliorating dry eye and symptoms thereof. In some embodiments, the formulations and methods provide for lubricating, hydrating, and/or nourishing ocular surface tissues.

In some embodiments, the formulation comprises at least one natural humectant, at least one amino acid, and at least one vitamin, wherein the formulation is preservative free. In some embodiments, the formulation comprises at least one natural humectant. In some embodiments, the formulation comprises at least one amino acid. In some embodiments, the formulation comprises at least one vitamin. In some embodiments, the formulation is preservative free. In some embodiments, the formulation comprises at least one natural humectant and is preservative free. In some embodiments, the formulation comprises at least one amino acid and is preservative free. In some embodiments, the formulation comprises at least one vitamin and is preservative free.

In some embodiments, the formulation comprises at least one natural humectant, at least one amino acid, and at least one vitamin, wherein the formulation is borate free. In some embodiments, the formulation comprises at least one natural humectant and is borate free. In some embodiments, the formulation comprises at least one amino acid and is borate free. In some embodiments, the formulation comprises at least one vitamin and is borate free

In some embodiments, the formulation is free of polyethylene glycol (PEG) and PEG-containing ingredients. In some embodiments, the formulation is free of polyethylene glycol (PEG). In some embodiments, the formulation is free of PEG-containing ingredients.

In some embodiments, the formulation comprises at least one natural humectant chosen from glycerin, trehalose, sucrose, and sodium hyaluronate. In some embodiments, the formulation comprises glycerin. In some embodiments, the formulation comprises trehalose. In some embodiments, the formulation comprises sucrose. In some embodiments, the formulation comprises sodium hyaluronate. In some embodiments, the formulation comprises glycerin and trehalose. In some embodiments, the formulation comprises glycerin and sodium hyaluronate. In some embodiments, the formulation comprises trehalose and sodium hyaluronate. In some embodiments, the formulation comprises sucrose and glycerin. In some embodiments, the formulation comprises sucrose and trehalose. In some embodiments, the formulation comprises sucrose and sodium hyaluronate. In some embodiments, the formulation comprises glycerin, trehalose, and sodium hyaluronate. In some embodiments, the formulation comprises glycerin, trehalose, sucrose, and sodium hyaluronate.

In some embodiments, the sodium hyaluronate is a high molecular weight sodium hyaluronate. In some embodiments, the sodium hyaluronate has a molecular weight in the range of 700 kDa to 1500 kDa. In some embodiments, the sodium hyaluronate has a molecular weight of 700 kDa. In some embodiments, the sodium hyaluronate has a molecular weight of 750 kDa. In some embodiments, the sodium hyaluronate has a molecular weight of 800 kDa. In some embodiments, the sodium hyaluronate has a molecular weight of 850 kDa. In some embodiments, the sodium hyaluronate has a molecular weight of 900 kDa. In some embodiments, the sodium hyaluronate has a molecular weight of 950 kDa. In some embodiments, the sodium hyaluronate has a molecular weight of 1000 kDa. In some embodiments, the sodium hyaluronate has a molecular weight of 1050 kDa. In some embodiments, the sodium hyaluronate has a molecular weight of 1100 kDa. In some embodiments, the sodium hyaluronate has a molecular weight of 1150 kDa. In some embodiments, the sodium hyaluronate has a molecular weight of 1200 kDa. In some embodiments, the sodium hyaluronate has a molecular weight of 1250 kDa. In some embodiments, the sodium hyaluronate has a molecular weight of 1300 kDa. In some embodiments, the sodium hyaluronate has a molecular weight of 1350 kDa. In some embodiments, the sodium hyaluronate has a molecular weight of 1400 kDa. In some embodiments, the sodium hyaluronate has a molecular weight of 1450 kDa. In some embodiments, the sodium hyaluronate has a molecular weight of 1500 kDa.

In some embodiments, the formulation comprises at least one amino acid chosen from arginine, leucine, taurine, proline, lysine, and methionine. In some embodiments, the formulation comprises arginine. In some embodiments, the formulation comprises leucine. In some embodiments, the formulation comprises taurine. In some embodiments, the formulation comprises proline. In some embodiments, the formulation comprises lysine. In some embodiments, the formulation comprises methinonine. In some embodiments, the formulation comprises arginine and leucine. In some embodiments, the formulation comprises arginine and taurine. In some embodiments, the formulation comprises leucine and taurine. In some embodiments, the formulation comprises arginine, leucine, and taurine.

In some embodiments, the formulation comprises at least one vitamin chosen from vitamin C and vitamin B₁₂. In some embodiment, the formulation comprises vitamin C and vitamin B₁₂.

In some embodiments, the formulation comprises vitamin C. In some embodiments, the vitamin C is chosen from sodium ascorbyl phosphate, magnesium ascorbyl phosphate, and combination thereof. In some embodiments, the vitamin C is sodium ascorbyl phosphate. In some embodiments, the vitamin C is magnesium ascorbyl phosphate. In some embodiments, the vitamin C is sodium ascorbyl phosphate and magnesium ascorbyl phosphate.

In some embodiments, the formulation comprises vitamin B₁₂. In some embodiments, the vitamin B₁₂ is chosen from cyanocobalamin, hydroxycobalamin, adenosyl cobalamin, methyl cobalamin, and combinations thereof. In some embodiments, the vitamin Biz is chosen from cyanocobalamin, hydroxycobalamin, adenosyl cobalamin, and methyl cobalamin. In some embodiments, the vitamin Biz is cyanocobalamin. In some embodiments, the vitamin B₁₂ is hydroxycobalamin. In some embodiments, the vitamin Biz is adenosyl cobalamin. In some embodiments, the vitamin Biz is methyl cobalamin. In some embodiments, the vitamin B₁₂ is cyanocobalamin and hydroxycobalamin. In some embodiments, the vitamin B₁₂ is cyanocobalamin and adenosyl cobalamin. In some embodiments, the vitamin B₁₂ is cyanocobalamin and methyl cobalamin. In some embodiments, the vitamin B₁₂ is hydroxycobalamin and adenosyl cobalamin. In some embodiments, the vitamin B₁₂ is hydroxycobalamin and methyl cobalamin. In some embodiments, the vitamin B₁₂ is adenosyl cobalamin and methyl cobalamin.

In some embodiments, the formulation comprises zinc and/or copper. In some embodiments, the formulation comprises zinc and copper. In some embodiments, the formulation comprises copper. In some embodiments, the formulation comprises zinc. In some embodiments, the amount of copper is below 5 ppm.

In some embodiments, the formulation further comprises at least one natural electrolyte. In some embodiments, the at least one natural electrolyte is chosen from sodium chloride, potassium chloride, calcium chloride, and magnesium chloride. In some embodiments, the formulation further comprises sodium chloride, potassium chloride, calcium chloride and magnesium chloride. In some embodiments, the formulation further comprises sodium chloride. In some embodiments, the formulation further comprises potassium chloride. In some embodiments, the formulation further comprises calcium chloride. In some embodiments, the formulation further comprises magnesium chloride. In some embodiments, the formulation further comprises sodium chloride and potassium chloride. In some embodiments, the formulation further comprises sodium chloride and calcium chloride. In some embodiments, the formulation further comprises sodium chloride and magnesium chloride. In some embodiments, the formulation further comprises potassium chloride and calcium chloride. In some embodiments, the formulation further comprises potassium chloride and magnesium chloride. In some embodiments, the formulation further comprises calcium chloride and magnesium chloride.

In some embodiments, the formulation further comprises at least one natural buffer agent. In some embodiments, the at least one natural buffer agent is chosen from sodium citrate, citric acid, sodium bicarbonate, and amino acids. In some embodiments, the formulation further comprises sodium citrate, citric acid, sodium bicarbonate and amino acids. In some embodiments, the formulation further comprises sodium citrate. In some embodiments, the formulation further comprises citric acid. In some embodiments, the formulation further comprises sodium bicarbonate. In some embodiments, the formulation further comprises sodium citrate and citric acid. In some embodiments, the formulation further comprises sodium citrate and sodium bicarbonate. In some embodiments, the formulation further comprises sodium citrate and amino acids. In some embodiments, the formulation further comprises citric acid and sodium bicarbonate. In some embodiments, the formulation further comprises citric acid and amino acids. In some embodiments, the formulation further comprises sodium bicarbonate and amino acids.

In some embodiments, the pH of the formulation is in a range of 6 to 8. In some embodiments the pH of the formulation is in a range of 6 to 7. In some embodiments the pH of the formulation is in a range of 7 to 8. In some embodiments the pH of the formulation is 6.0. In some embodiments the pH of the formulation is 6.5. In some embodiments the pH of the formulation is 7.0. In some embodiments the pH of the formulation is 7.5. In some embodiments the pH of the formulation is 8.0.

In some embodiments, the formulation has an osmolality of 200 mOsm/kg to 300 mOsm/kg. In some embodiments, the formulation has an osmolality of 200 mOsm/kg. In some embodiments, the formulation has an osmolality of 215 mOsm/kg. In some embodiments, the formulation has an osmolality of 230 mOsm/kg. In some embodiments, the formulation has an osmolality of 245 mOsm/kg. In some embodiments, the formulation has an osmolality of 275 mOsm/kg. In some embodiments, the formulation has an osmolality of 300 mOsm/kg.

In some embodiments, the formulation comprises, glycerin, trehalose, sodium hyaluronate, sodium hyaluronate, sodium chloride, potassium chloride, magnesium ascorbyl phosphate, sodium citrate, L-arginine, citric acid, L-taurine, L-leucine, calcium chloride, sodium bicarbonate, magnesium chloride, cyanocobalamin, and sodium hydroxide.

In some embodiments, the formulation comprises 0.6% w/v to 0.8% w/v glycerin, 0.25% w/v to 0.5% w/v trehalose, 0.1% w/v to 0.3% w/v sodium hyaluronate, 0.2% w/v to 0.3% w/v sodium chloride, 0.01% w/v to 0.14% w/v potassium chloride, 0.01% w/v to 0.1% w/v magnesium ascorbyl phosphate, 0.02% w/v to 0.06% w/v sodium citrate, 0.02% w/v to 0.05% w/v L-arginine, 0.01% w/v to 0.0275% w/v citric acid, 0.01% w/v to 0.02% w/v L-taurine, 0.01% w/v to 0.02% w/v L-leucine, 0.01% w/v to 0.02% w/v calcium chloride, 0.010% w/v to 0.015% w/v sodium bicarbonate, 0.005% w/v to 0.011% w/v magnesium chloride, 0.001% w/v to 0.004% w/v cyanocobalamin, and qs % w/v sodium hydroxide.

In some embodiments, the formulation comprises 0.75% w/v glycerin, 0.5% w/v trehalose, 0.24% w/v sodium hyaluronate, 0.3% w/v sodium chloride, 0.14% w/v potassium chloride, 0.1% w/v magnesium ascorbyl phosphate, 0.06% w/v sodium citrate, 0.05% w/v L-arginine, 0.0275% w/v citric acid. 0.02% w/v L-taurine. 0.02% w/v L-leucine, 0.02% w/v calcium chloride, 0.015% w/v sodium bicarbonate, 0.011% w/v magnesium chloride, 0.004% w/v cyanocobalamin, and qs % w/v sodium hydroxide.

In some embodiments, the formulations disclosed herein are stable. In some embodiments, the formulations are shelf-stable.

In some embodiments, after being stored at 20-25° C. for 24 months, the pH of the formulation does not drop more than 0.75 pH units. In some embodiments, after being stored at 20-25° C. for 24 months, the pH of the formulation does not drop more than 0.6 pH units. In some embodiments, after being stored at 20-25° C. for 24 months, the pH of the formulation does not drop more than 0.5 pH units. In some embodiments, after being stored at 20-25° C. for 24 months, the pH of the formulation does not drop more than 0.4 pH units. In some embodiments, after being stored at 20-25° C. for 24 months, the pH of the formulation does not drop more than 0.3 pH units. In some embodiments, after being stored at 20-25° C. for 24 months, the pH of the formulation does not drop more than 0.25 pH units. In some embodiments, after being stored at 20-25° C. for 24 months, the pH of the formulation does not drop more than 0.2 pH units. In some embodiments, after being stored at 20-25° C. for 24 months, the pH of the formulation does not drop more than 0.1 pH units.

In some embodiments, after being stored at 20-25° C. for 24 months, the osmolality of the formulation does not increase more than 50 mOsm. In some embodiments, after being stored at 20-25° C. for 24 months, the osmolality of the formulation does not increase more than 40 mOsm. In some embodiments, after being stored at 20-25° C. for 24 months, the osmolality of the formulation does not increase more than 30 mOsm. In some embodiments, after being stored at 20-25° C. for 24 months, the osmolality of the formulation does not increase more than 25 mOsm. In some embodiments, after being stored at 20-25° C. for 24 months, the osmolality of the formulation does not increase more than 20 mOsm. In some embodiments, after being stored at 20-25° C. for 24 months, the osmolality of the formulation does not increase more than 15 mOsm.

In some embodiments, the formulation is packaged in a sealed package. In some embodiments, the formulation is sealed in a plastic bottle. In some embodiments, the plastic bottle is made of polyethylene. In some embodiments, the plastic bottle is made of polypropylene. In some embodiments, the plastic bottle is made of polyethylene terephthalate (PET).

In some embodiments, the formulation is sterilized by filtration, autoclaving, gamma irradiation and/or electron-beam. In some embodiments, the formulation is sterilized by filtration. In some embodiments, the formulation is sterilized by autoclaving. In some embodiments, the formulation is sterilized by filtration and autoclaving. In some embodiments, the formulation is sterilized by gamma irradiation. In some embodiments, the formulation is sterilized by electron-beam.

In some embodiments, the formulations disclosed herein are free of parabens, thiazolinones, and formaldehyde-donor preservatives. In some embodiments, formulations disclosed herein are free of polyethylene glycol (PEG) and PEG-containing ingredients. In some embodiments, formulations disclosed herein are preservative free. In some embodiments, formulations disclosed herein are sterile and preservative free. In some embodiments, the formulations disclosed herein are borate free. In some embodiments, formulations disclosed herein are pH-balanced and non-irritating.

The formulations disclosed herein may be prepared according to any known method for the manufacture of cosmetic formulations or preparations. As will be appreciated by those of ordinary skill in the art, a number of methods are known.

In some embodiments, the formulations disclosed herein are useful for cleansing, cleaning, conditioning, moisturizing, lubricating, hydrating, nourishing, soothing, calming, and/or refreshing, ocular surface tissues. In some embodiments, formulations disclosed herein are useful for treating, improving, and/or ameliorating dryness; removing irritants and/or debris; maintaining eye hygiene; and/or treating, improving, and/or ameliorating dry eye and symptoms thereof. In some embodiments, formulations disclosed herein are suitable for adults, children, and contact-lens wearers.

Accordingly, provided herein are methods of cleansing, cleaning, conditioning, moisturizing, lubricating, hydrating, nourishing, soothing, calming, and/or refreshing ocular surface tissues, comprising applying to said ocular surface tissues a formulation as disclosed herein. In some embodiments, provided herein are methods of treating, improving, and/or ameliorating dryness; removing irritants and/or debris; maintaining eye hygiene; and/or treating, improving, or ameliorating dry eye and symptoms thereof. The methods comprise applying to ocular surface tissues a formulation as disclosed herein.

### EXAMPLES

### Example 1: Exemplary Formulation

Formulations of the present disclosure can be prepared with ingredients as listed in the following Formulations of the present disclosure can be prepared with ingredients as listed in the following table.

TABLE 1

| Ingredient | % w/v |
|---|---|
| Glycerin | 0.750 |
| Trehalose | 0.500 (13 mmol/L) |
| Sodium hyaluronate | 0.240 |
| Sodium chloride | 0.300 |
| Potassium chloride | 0.140 (18.8 mmol/L) |
| Magnesium ascorbyl phosphate | 0.100 (3.2 mmol/L) |
| Sodium citrate | 0.060 (2 mmol/L) |
| L-arginine | 0.050 (2.9 mmol/L) |
| Citric acid | 0.024 (1.4 mmol/L) |
| L-Taurine | 0.020 (1.6 mmol/L) |
| L-Leucine | 0.020 (1.6 mmol/L) |
| Calcium chloride | 0.020 (1.36 mmol/L) |
| Sodium bicarbonate | 0.005 (1.8 mmol/L) |
| Cyanocobalamin (B₁₂) | 0.004 |
| Purified water | Q.S. to 100% volume |

Formulations with the ingredients listed above can be prepared by mixing the recited ingredients, for example according to the proposed manufacturing process below:
1. In an appropriate mixing vessel, add volume of water equivalent to 80% of the total batch weight.
2. Sprinkle in sodium hyaluronate while mixing, until the polymer is fully hydrated, NLT 1 hour to ensure homogenous dispersing.
3. Slowly add batch quantities of the remaining ingredients to the solution. Mix NLT 30 minutes to ensure complete dissolution. For example, one order of addition could be: glycerin, trehalose, sodium chloride, potassium chloride, magnesium chloride, calcium chloride, magnesium ascorbyl phosphate, sodium citrate, citric acid, sodium bicarbonate, amino acids, and vitamin B12 (cyanocobalamin).
4. Q.S. to 100% batch weight with purified water.
5. Sterile filter the formulation with an appropriate sterilizing filter (0.2 μm PES).

The pH, osmolality, and color of the formulations are assessed.

A pH adjuster could be added. For example, sodium hydroxide and/or hydrochloric acid may be added to adjust pH. As a non-limiting example, 1N sodium hydroxide could be added to obtain a pH of 7.5.

Other key ingredients could be added. For example, at least one amino acid chosen from L-proline, L-lysine, and L-methionine, or combinations thereof. Propylene glycol could also be used as an additional humectant, and sucrose could be used as an alternative saccharide. Copper and/or zinc could be added as an additional nutritional component. These metals are added to vitamin supplements and operate as vitamin co-factors. For example, copper could be added in an amount of about 5 ppm and zinc could be added at higher levels.

### Example 2: Prophetic Formulations

Other prophetic, exemplary formulations of the present disclosure are listed in the following Table 2.

TABLE 2

| Ingredient | % w/v | % w/v | % w/v | % w/v | % w/v | % w/v | % w/v | % w/v | % w/v | % w/v |
|---|---|---|---|---|---|---|---|---|---|---|
| Glycerin | 0.750 | 0.750 | 1.000 | 0.500 | 1.000 | 0.750 | 0.750 | 1.000 | 0.500 | 1.000 |
| Propylene Glycol | — | — | — | 0.500 | — | — | — | — | 0.500 | — |
| Trehalose | 0.500 | 0.500 | — | 0.250 | 0.500 | 0.500 | 0.500 | — | 0.250 | 0.500 |
| Sucrose | — | — | 0.500 | 0.250 | — | — | — | 0.500 | 0.250 | — |
| Sodium Hyaluronate | 0.240 | 0.240 | 0.240 | 0.240 | 0.240 | 0.1 | 0.15 | 0.1 | 0.2 | 0.15 |
| Sodium Chloride | 0.300 | 0.300 | 0.300 | 0.300 | 0.300 | 0.300 | 0.300 | 0.300 | 0.300 | 0.300 |
| Potassium Chloride | 0.140 | 0.140 | 0.140 | 0.140 | 0.140 | 0.140 | 0.140 | 0.140 | 0.140 | 0.140 |
| Sodium Ascorbyl | — | 0.100 | — | — | — | — | 0.100 | — | — | — |
| Phosphate | | | | | | | | | | |
| Magnesium Ascorbyl | 0.100 | — | 0.100 | 0.100 | 0.100 | 0.100 | — | 0.100 | 0.100 | 0.100 |
| Phosphate | | | | | | | | | | |
| Sodium Citrate | 0.060 | 0.060 | 0.060 | 0.060 | 0.060 | 0.060 | 0.060 | 0.060 | 0.060 | 0.060 |
| Citric Acid Anhydrous | 0.024 | 0.0275 | 0.024 | 0.024 | 0.024 | 0.024 | 0.0275 | 0.024 | 0.024 | 0.024 |
| Calcium Chloride | 0.020 | 0.020 | 0.020 | 0.020 | 0.020 | 0.020 | 0.020 | 0.020 | 0.020 | 0.020 |
| Magnesium Chloride | — | 0.011 | — | — | — | — | 0.011 | — | — | — |
| Zinc Chloride | — | — | — | — | 0.0015 | — | — | — | — | 0.0015 |
| Copper Gluconate | — | — | — | 0.0020 | — | — | — | — | 0.0020 | — |
| L-Arginine (Base) | 0.050 | 0.050 | 0.050 | 0.050 | 0.050 | 0.050 | 0.050 | 0.050 | 0.050 | 0.050 |
| Taurine | 0.020 | 0.020 | 0.020 | 0.020 | 0.020 | 0.020 | 0.020 | 0.020 | 0.020 | 0.020 |
| L-Leucine | 0.020 | 0.020 | 0.020 | 0.020 | 0.020 | 0.020 | 0.020 | 0.020 | 0.020 | 0.020 |
| L-Proline | — | — | 0.020 | 0.050 | 0.050 | — | — | 0.020 | 0.050 | 0.050 |
| L-lysine | — | — | 0.020 | — | — | — | — | 0.020 | — | — |
| L-methionine | — | — | 0.020 | — | — | — | — | 0.020 | — | — |
| Cyanocobalamin (B12) | 0.004 | 0.004 | 0.004 | 0.004 | 0.004 | 0.004 | 0.004 | 0.004 | 0.004 | 0.004 |
| Sodium Bicarbonate | 0.005 | 0.015 | 0.005 | 0.005 | 0.005 | 0.005 | 0.015 | 0.005 | 0.005 | 0.005 |
| Sodium Hydroxide (1N) | 0.046 | 0.046 | 0.046 | 0.046 | 0.046 | 0.046 | 0.046 | 0.046 | 0.046 | 0.046 |
| Purified Water | Q.S. | Q.S. | Q.S. | Q.S. | Q.S. | Q.S. | Q.S. | Q.S. | Q.S. | Q.S. |
| | to 100 | to 100 | to 100 | to 100 | to 100 | to 100 | to 100 | to 100 | to 100 | to 100 |

### Example 3: A Study to Test the Effects of Vitamin Lubricating PF Drops on Ocular Irritation

Purpose: To evaluate the ocular irritation of the preservative-free, vitamin lubricating eye drop formulation of the present disclosure (“Vitamin Lubricating PF”) packaged in PET bottle, following topical ocular administration to New Zealand white rabbits 6 times daily for 5 days.

Formulations: The Test Article employed has a formulation according to Table 1 above.

Methods: 60 μL, of the Test Article is instilled into the right eye of each animal via micropipette, six times per day at 1.5 hour±15 minute intervals. Instillation was made in the cup formed after the lower eyelid (conjunctival sac) was gently pulled away from the globe (eyeball). Lids were held together for approximately one second after instillation. The left eye remained untreated as a control.

On Day 1 prior to the first dose administration, all eyes were examined microscopically via slit lamp with fluorescein stain. Eyes were also examined macroscopically using the Draize scoring system. Both examinations confirmed that all animals had clinically normal eyes prior to initiation of dosing.

The animals' eyes were examined three times daily using the Draize Method. Scoring was performed just prior to the first dose instillation, approximately 10 minutes after the third dose instillation, and a minimum of ten minutes after the sixth dose instillation. Positive or negative irritation scores were as follows.

Positive irritation score: Scores of >1 for conjunctival redness, >1 for conjunctival chemosis, >0 for corneal opacity, and/or >0 for iris involvement. A positive irritation score was not considered clinically significant.

Negative irritation score: Scores of ≤1 for conjunctival redness, ≤1 for conjunctival chemosis, 0 for corneal opacity, and/or 0 for iris involvement.

On Day 5, after a minimum of 10 minutes after the last dose instillation, the animals were examined microscopically using slit lamp with fluorescein stain as previously described.

Results

All results indicate that the preservative-free, vitamin lubricating eye drop formulation of the present disclosure did not elicit a positive irritation response. There were no scores for irritation observed in either the right (test) or left (control) eyes at any observation point throughout the study.

TABLE 3
Individual Microscopic Ocular Examinations - McDonald Shadduck Scoring

| | | | | | | | Cornea | | | | |
|---|---|---|---|---|---|---|---|---|---|---|---|
| | | Conjunctiva | | | Aqueous | Iris | Severity of | Area of | | Fluorescein | Lens |
| Animal | Eye | Congestion | Swelling | Discharge | flare | Involvement | Opacity | Opacity | Pannus | Stain | (N or A) |

Pre-dose Observations

| | | | | | | | | | | | |
|---|---|---|---|---|---|---|---|---|---|---|---|
| 1 | R | 0 | 0 | 0 | 0 | 0 | 0 | 0 | 0 | 0 | N |
| | L | 0 | 0 | 0 | 0 | 0 | 0 | 0 | 0 | 0 | N |
| 2 | R | 0 | 0 | 0 | 0 | 0 | 0 | 0 | 0 | 0 | N |
| | L | 0 | 0 | 0 | 0 | 0 | 0 | 0 | 0 | 0 | N |
| 3 | R | 0 | 0 | 0 | 0 | 0 | 0 | 0 | 0 | 0 | N |
| | L | 0 | 0 | 0 | 0 | 0 | 0 | 0 | 0 | 0 | N |
| 4 | R | 0 | 0 | 0 | 0 | 0 | 0 | 0 | 0 | 0 | N |
| | L | 0 | 0 | 0 | 0 | 0 | 0 | 0 | 0 | 0 | N |
| 5 | R | 0 | 0 | 0 | 0 | 0 | 0 | 0 | 0 | 0 | N |
| | L | 0 | 0 | 0 | 0 | 0 | 0 | 0 | 0 | 0 | N |

Day 5, Post-dose

| | | | | | | | | | | | |
|---|---|---|---|---|---|---|---|---|---|---|---|
| 1 | R | 0 | 0 | 0 | 0 | 0 | 0 | 0 | 0 | 0 | N |
| | L | 0 | 0 | 0 | 0 | 0 | 0 | 0 | 0 | 0 | N |
| 2 | R | 0 | 0 | 0 | 0 | 0 | 0 | 0 | 0 | 0 | N |
| | L | 0 | 0 | 0 | 0 | 0 | 0 | 0 | 0 | 0 | N |
| 3 | R | 0 | 0 | 0 | 0 | 0 | 0 | 0 | 0 | 0 | N |
| | L | 0 | 0 | 0 | 0 | 0 | 0 | 0 | 0 | 0 | N |
| 4 | R | 0 | 0 | 0 | 0 | 0 | 0 | 0 | 0 | 0 | N |
| | L | 0 | 0 | 0 | 0 | 0 | 0 | 0 | 0 | 0 | N |
| 5 | R | 0 | 0 | 0 | 0 | 0 | 0 | 0 | 0 | 0 | N |
| | L | 0 | 0 | 0 | 0 | 0 | 0 | 0 | 0 | 0 | N |

N: Normal;
A: Abnormal

The McDonald Shadduck score system is outlined below.

Conjunctival Congestion
0=Normal. May appear blanched to reddish pink without perilimbal injection (except at 12:00 and 6:00 o'clock positions) with vessels of the palpebral and bulbar conjunctiva easily observed.
1=A flushed, reddish color predominately confined to the palpebral conjunctiva with some perilimbal injection but primarily confined to the lower and upper parts of the eye from the 4:00 and 7:00 and 11:00 to 1:00 o'clock positions.
2=Bright red color of the palpebral conjunctiva with accompanying perilimbal injection covering at least 75% of the circumference of the perilimbal region.
3=Dark, beefy red color with congestion of both the bulbar and the palpebral conjunctiva along with pronounced perilimbal injection and the presence of petechia on the conjunctiva. The petechia generally predominate along the nictitating membrane.

Conjunctival Swelling
0=Normal or no swelling of the conjunctival tissue.
1=Swelling above normal without eversion of the lids (can be easily ascertained by noting that the upper and lower eyelids are positioned as in the normal eye); swelling generally starts in the lower cul-de-sac near the inner canthus, which needs slit-lamp examination.
2=Swelling with misalignment of the normal approximation of the lower and upper eyelids; primarily confined to the upper eyelid so that in the initial stages the misapproximation of the eyelids begins by partial eversion of the upper eyelid. In this stage, swelling is confined generally to the upper eyelid, although it exists in the lower cul-de-sac (observed best with the slit-lamp).
3=Definite swelling with partial eversion of the upper and lower eyelids essentially equivalent. This can be easily ascertained by looking at the animal head-on and noticing the positioning of the eyelids; if the eye margins do not meet, eversion has occurred.
4=Eversion of the upper eyelids is pronounced with less pronounced eversion of the lower eyelid. It is difficult to retract the lids and observe the perilimbal region.

Conjunctival Discharge

Discharge is defined as a whitish, gray precipitate, which should not be confused with the small amount of clear, inspissated, mucoid material that can be formed in the medial canthus of a substantial number of rabbit eyes.
0=Normal. No discharge.
1=Discharge above normal and present on the inner portion of the eye but not on the lids or hairs of the eyelids. One can ignore the small amount that is in the inner and outer canthus.
2=Discharge is abundant, easily observed, and has collected on the lids and around the hairs of the eyelids.
3=Discharge has been flowing over the eyelids so as to wet the hairs substantially on the skin around the eye.

Aqueous Flare

The intensity of the Tyndall phenomenon is scored by comparing the normal Tyndall effect observed when the slit-lamp beam passes through the lens with that seen in the anterior chamber. The presence of aqueous flare is presumptive evidence of breakdown of the blood-aqueous barrier.
0=The absence of visible light beam light in the anterior chamber (no Tyndall effect).
1=The Tyndall effect is barely discernible. The intensity of the light beam in the anterior chamber is less than the intensity of the slit beam as it passes through the lens.
2=The Tyndall beam in the anterior chamber is easily discernible and is equal in intensity to the slit beam as it passes through the lens.
3=The Tyndall beam in the anterior chamber is easily discernible; its intensity is greater than the intensity of the slit beam as it passes through the lens.

Iris INVOLVEMENT

In the following definitions the primary, secondary, and tertiary vessels are utilized as an aid to determining a subjective ocular score for iris involvement. The assumption is made that the greater the hyperemia of the vessels and the more the secondary and tertiary vessels are involved, the greater the intensity of iris involvement.
0=Normal iris without any hyperemia of the iris vessels. Occasionally around the 12:00 to 1:00 o'clock position near the pupillary border and the 6:00 to 7:00 o'clock position near the pupillary border there is a small area around 1-3 mm in diameter in which both the secondary and tertiary vessels are slightly hyperemic.
1=Minimal injection of secondary vessels but not tertiary. Generally, it is uniform, but may be of greater intensity at the 1:00 or 6:00 o'clock position. If it is confined to the 1:00 or 6:00 o'clock position, the tertiary vessels must be substantially hyperemic.
2=Minimal injection of tertiary vessels and minimal to moderate injection of the secondary vessels.
3=Moderate injection of the secondary and tertiary vessels with slight swelling of the iris stroma (this gives the iris surface a slightly rugose appearance, which is usually most prominent near the 3:00 and 9:00 o'clock positions).
4=Marked injection of the secondary and tertiary vessels with marked swelling of the iris stroma. The iris appears rugose; may be accompanied by hemorrhage (hyphema) in the anterior chamber.

Cornea

The scoring scheme measures the severity of corneal cloudiness and the area of the cornea involved. Severity of corneal cloudiness is graded as follows:
0=Normal cornea. Appears with the slit-lamp as having a bright gray line on the epithelial surface and a bright gray line on the endothelial surface with a marble-like gray appearance of the stroma.
1=Some loss of transparency. Only the anterior half of the stroma is involved as observed with an optical section of the slit-lamp. The underlying structures are clearly visible with diffuse illumination, although some cloudiness can be readily apparent with diffuse illumination.
2=Moderate loss of transparency. In addition to involving the anterior stroma, the cloudiness extends all the way to the endothelium. The stroma has lost its marble-like appearance and is homogeneously white. With diffuse illumination, underlying structures are clearly visible.
3=Involvement of the entire thickness of the stroma. With optical section, the endothelial surface is still visible. However, with diffuse illumination the underlying structures are just barely visible (to the extent that the observer is still able to grade flare, iritis, observe for pupillary response, and note lenticular changes).
4=Involvement of the entire thickness of the stroma. With the optical section, cannot clearly visualize the endothelium. With diffuse illumination, the underlying structures cannot be seen. Cloudiness removes the capability for judging and grading aqueous flare, iritis, lenticular changes, and pupillary response.
   The surface area of the cornea relative to the area of cloudiness is divided into five grades from 0 to 4.
0=Normal cornea with no area of cloudiness.
1=1-25% area of stromal cloudiness.
2=26-50% area of stromal cloudiness.
3=51-75% area of stromal cloudiness.
4=76-100% area of stromal cloudiness.

Pannus

Pannus is vascularization or the penetration of new blood vessels into the corneal stroma. The vessels are derived from the limbal vascular loops. Pannus is divided into three grades.
0=No pannus.
1=Vascularization is present but vessels have not invaded the entire corneal circumference. Where localized vessel invasion has occurred, they have not penetrated beyond 2 mm.
2=Vessels have invaded 2 mm or more around the entire corneal circumference.

Fluorescein

The use of fluorescein is a valuable aid in defining epithelial damage. The area of staining can be judged on a 0 to 4 scale using the same terminology as for corneal cloudiness.
0=Absence of fluorescein staining.
1=Slight fluorescein staining confined to a small focus. With diffuse illumination the underlying structures are easily visible. (The outline of the pupillary margin is as if there were no fluorescein staining.)
2=Moderate fluorescein staining confined to a small focus. With diffuse illumination the underlying structures are clearly visible, although there is some loss of detail.
3=Marked fluorescein staining. Staining may involve a larger portion of the cornea. With diffuse illumination the underlying structures are barely visible but are not completely obliterated.
4=Extreme fluorescein staining. With diffuse illumination the underlying structures cannot be observed.

Lens

The lens should be evaluated routinely during ocular evaluations and graded as either normal (N) or abnormal (A).

TABLE 4
Individual Macroscopic Ocular Examinations - Gross Ocular Observations (Draize)

| | Cornea | | | | | | Iris | | | | Conjunctiva | | | | | | | | | |
|---|---|---|---|---|---|---|---|---|---|---|---|---|---|---|---|---|---|---|---|---|
| | Opacity | | Area | | Subtotal | | Score | | Subtotal | | Redness | | Chemosis | | Discharge | | Subtotal | | Total | |
| | (A) | | (B) | | A × B × 5= | | (A) | | A × 5= | | (A) | | (B) | | (B) | | (A + B + C) × 2 | | Score | |
| Animal | L | R | L | R | L | R | L | R | L | R | L | R | L | R | L | R | L | R | L | R |

Day 1, Pre-dose #1

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 1, 10 Minutes Post-dose #3

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 1, 10 Minutes Post-dose #6

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 2, Pre-dose #1

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 2, 10 Minutes Post-dose #3

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 2, 10 Minutes Post-dose #6

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 3, Pre-dose #1

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 3, 10 Minutes Post-dose #3

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 3, 10 Minutes Post-dose #6

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 4, Pre-dose #1

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 4, 10 Minutes Post-dose #3

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 4, 10 Minutes Post-dose #6

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 5, Pre-dose #1

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 5, 10 Minutes Post-dose #3

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

Day 5, 10 Minutes Post-dose #6

| | | | | | | | | | | | | | | | | | | | | |
|---|---|---|---|---|---|---|---|---|---|---|---|---|---|---|---|---|---|---|---|---|
| 1 | 0 | 0 | 0 | 0 | 0 | 0 | 0 | 0 | 0 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 | 0 | 0 | 0 | 0 | 0 | 0 | 0 | 0 | 0 |
| 5 | 0 | 0 | 0 | 0 | 0 | 0 | 0 | 0 | 0 | 0 | 0 | 0 | 0 | 0 | 0 | 0 | 0 | 0 | 0 | 0 |

### Example 4: A Study to Test the Effects of Dry Eye Products on Metabolic Activity and Cytokine Response in ATCC Human Corneal Epithelial Cells Exposed to Hyperosmolarity

Purpose: To determine the cell metabolic activity and proinflammatory cytokine/chemokine response (IL-6, IL-8 and TNF-α) of ATCC-HCEC exposed to 25% over-the-counter dry eye drops (OTC-DED) solutions and the preservative free vitamin formulation of the present disclosure in 75% EpiLife medium and each solution adjusted with NaCl to increase the osmolarity to 400 and 450 mOsm/kg.

Prior studies indicated that exposure of a transformed human corneal epithelial cell line (ATCC-HCEC) to hyperosmolar medium containing 0.9% erythritol and 0.9% glycerol (Matsuo, Br. J. Ophthalmol., 85:610-612, 2001) and Infuse packaging solution (Paulsen et al., Med. Sci. Monit., 14:PI12-16, 2008) containing both erythritol and glycerol reduced the cellular release of pro-inflammatory cytokines when compared to cells not receiving the osmoprotectants. In the present study, preliminary investigations were initiated to evaluate the effects of ATCC-HCEC exposed to 25% solutions of the preservative free vitamin lubricating formulation of the present disclosure and commercially available aqueous and emulsion based over-the-counter dry eye drops (OTC-DED) products. In addition, these formulations were prepared as hyperosmolar solutions to evaluate their osmoprotective properties on metabolic activity and cytokine response in the ATCC-HCEC line.

Methods: ATCC-HCEC were seeded in a 96 well plate. After culture for 72 h, cell medium was changed, and cells were incubated for an additional 24 h. Cells were exposed to 75% EpiLife medium with either 25% HBSS as a control or 25% of each OTC-DED products, as shown in Table 5, below, for 24 hours. The preservative-free, vitamin lubricating eye drops formulation of the present disclosure (“Vitamin Lubricating PF”) was also tested.

TABLE 5
Formulation Ingredients of Over-the Counter Dry Eye Drops Products

| | Formulation | | | |
|---|---|---|---|---|
| | Systane Complete + | Systane Complete PF | Systane Hydration PF | Refresh Relieva PF |
| | Preservative | (Preservative Free) | (Preservative Free) | (Preservative Free) |
| Ingredients | Borate | Borate | Borate | Borate |
| | Sorbitol | NaCl | NaCl, KCl | Citrate |
| | Propylene Glycol, | Sorbitol | Sorbitol | KCl, CaCl₂ |
| | Dimyristoyl | Propylene Glycol, | Sodium Hyaluronate | MgCl₂ |
| | phosphatidylglycerol, | Dimyristoyl | Propylene Glycol, | HA |
| | Hydroxypropyl guar, | phosphatidylglycerol, | polyethylene glicol, | CMC |
| | Mineral oil, | Hydroxypropyl guar, | Aminomethyl- | Preservative |
| | Polyoxl 40 stearate, | Mineral oil, | Propanol, | Free |
| | Sorbitan tristearate | Polyoxl 40 stearate, | Hydroxypropyl guar | |
| | EDTA | Sorbitan tristearate | | |
| | 0.001% PQ-1 | | | |

All products were tested as described in Table 6, below. Each test formulation was adjusted with NaCl to 400 or 450 mOsm/kg and cells were incubated for 24 hours. Each exposure was performed in triplicate. Upon completion, the cell culture supernatant was collected and analyzed for release of cytokines IL-6 and TNF-α and chemokine IL-8 and cell metabolic activity was determined by the AlamarBlue™ assay, as described below.

TABLE 6
Experimental Design and Schedule Summary

| Day 1-3 | Day 4 treatment (24 hours) | |
|---|---|---|
| Initial ATCC-HCEC | 75% EpiLife:25% HBSS | |
| Culture Conditions | or dry eye drop product | Day 5 Analysis |
| Seed ATCC-HCEC | HBSS | Cells were washed 1 |
| 96 well plate and | Systane Complete (PF) | time with 0.2 ml |
| allow to grow a | Systane Complete + Preservative | DMEM and added 0.1 |
| minimum of 72 hours | Systane Hydration PF | ml of 10% alamarBlue |
| | Refresh Relieva PF | in DMEM for 3 hours |
| | Vitamin Lubricating PF | and fluorescence read |
| | HBSS (400 mOsm/kg) | with BioTek Synergy |
| | Systane Complete PF (400 mOsm/kg) | plate reader |
| | Systane Complete + Preservative | |
| | (400 mOsm/kg) | |
| | Systane Hydration PF (400 mOsm/kg) | |
| | Refresh Relieva PF (400 mOsm/kg) | |
| | Vitamin Lubricating PF (400 mOsm/kg) | |
| | HBSS (450 mOsm/kg) | |
| | Systane Complete PF (450 mOsm/kg) | |
| | Systane Complete + Preservative | |
| | (450 mOsm/kg) | |
| | Systane Hydration PF (450 mOsm/kg) | |
| | Refresh Relieva PF (450 mOsm/kg) | |
| | Vitamin Lubricating PF (450 mOsm/kg) | |

Cytokine Analysis: The cell supernatants were assayed using a Millipore Magpix kit for the detection of cytokines IL-6 and TNF-α and chemokine IL-8 and were quantitated using a Magpix kit generated standard curve to determine concentrations.

Metabolic Activity Analysis: ATCC-HCEC lines were assessed for metabolic activity, using AlamarBlue™. In brief, cells were washed 1× with 0.2 ml/well Dulbecco's Modified Eagle's medium (DMEM). The AlamarBlue™ solution was diluted in DMEM at 10% v/v, pre-warmed in a water bath, and 0.1 ml solution per well was added. Each plate was incubated for 3 hours at 37° C., 5% CO₂, and 95% relative humidity. Each plate was read on a fluorescent microplate reader and results were assayed using relative fluorescence units (RFUs).

Cell viability was assessed by comparing RFUs for each ATCC-HCEC test group to the HBSS control.

Results

Cytokine Responses: TNF-α responses were below the limit of detection and were not analyzed.

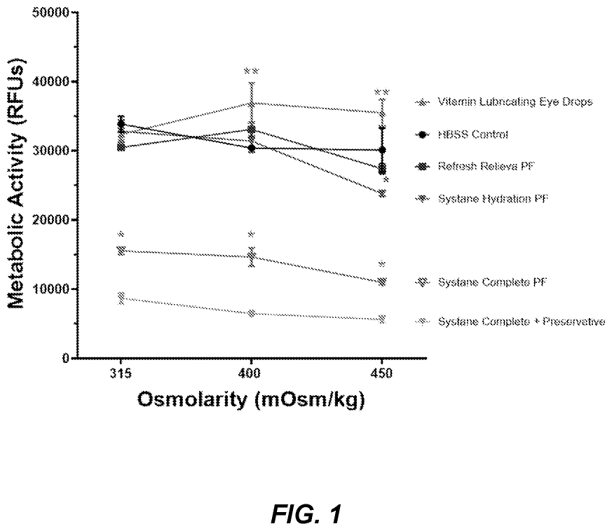

Metabolic Activity/Viability: There was significantly lower metabolic activity with Systane Hydration PF at 450 mOsm/kg and Systane Complete with and without preservative solutions at all 3 osmolarity conditions tested versus the HBSS control. (See FIG. 1). Significantly higher metabolic activity was observed with preservative free vitamin lubricating formulation of the present disclosure (“Vitamin Lubricating Eye Drops”) at 400 and 450 mOsm/kg (see FIG. 1).

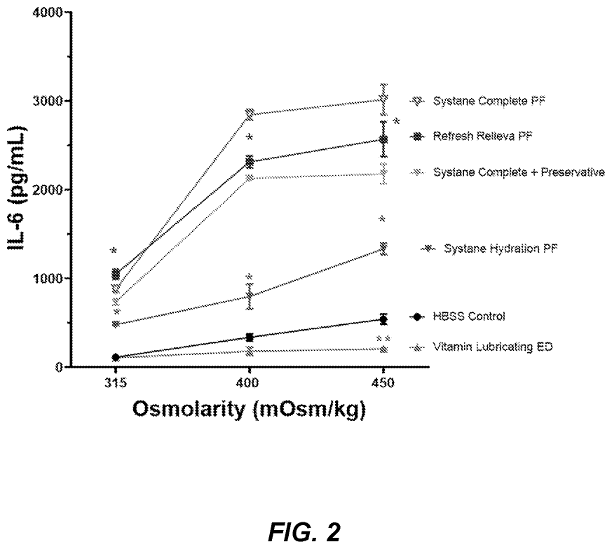

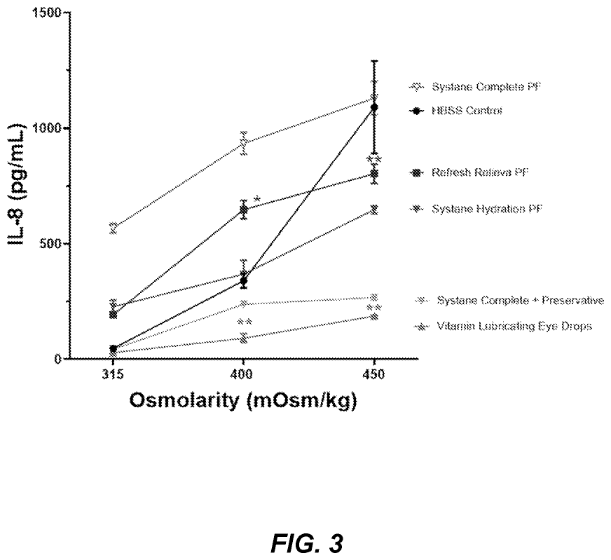

Statistical significance of cytokine IL-6 and chemokine IL-8 were not noted on graphs for all tests with the Systane Complete with Preservative, Systane Complete PF, and Systane Hydration PF at 450 mOsm/kg because of the significantly lower metabolic activity. The IL-6 and IL-8 responses for these three products are shown in FIGS. 2 and 3.

Refresh Relieva PF and Systane Hydration PF had significantly higher IL-6 responses at all 3 test conditions versus the HBSS control and the preservative free vitamin lubricating formulation of the present disclosure (“Vitamin Lubricating ED”) had a significantly lower IL-6 response at 450 mOsm/kg (see FIG. 2).

Refresh Relieva PF had significantly higher IL-8 response at 400 mOsm/kg and lower at 450 mOsm/kg versus the HBSS control (FIG. 3). Significantly lower IL-8 responses were found with the preservative free vitamin lubricating formulation of the present disclosure (“Vitamin Lubricating Eye Drops”) had at 400 and 450 mOsm/kg (see FIG. 3).

Of the currently marketed products tested in this study, the preservative free, aqueous based vitamin lubricating formulation of the present disclosure performed better than marketed products and controls by significantly reducing pro-inflammatory IL-6 and IL-8 responses.

### Example 5: A Study to Test the Effects of Dry Eye Products on Metabolic Activity and Cytokine Response in Riken Human Corneal Epithelial Cells Exposed to Hyperosmolarity

Purpose: To determine the cell metabolic activity and proinflammatory cytokine/chemokine response (IL-6, IL-8, and MCP-1) of Riken-HCEC exposed to 25% preservative free vitamin formulation of the present disclosure and compositional variations thereof in 75% DMEM/F12 medium and each solution adjusted with NaCl to increase the osmolarity to 400, 450, and 500 mOsm/kg.

Methods: Riken-HCEC were seeded in a 96 well plate. After culture for 72 h, cell medium was changed, and cells were incubated for an additional 24 h. Cells were exposed to 75% DMEM/F12 medium with either 25% HBSS as a control or 25% of each compositional variation of the preservative free vitamin formulation of the present disclosure, as shown in Table 7, below, for 24 hours. The preservative-free, vitamin lubricating eye drops formulation of the present disclosure was also tested.

TABLE 7
Ingredients of Tested Formulations

| | Full | Form. B | | Form. D | | | | |
|---|---|---|---|---|---|---|---|---|
| | Formulation | (Full Form. - | | (Full Form. - | Form. E | Form. F | | |
| | Vitamin | Glycerin - | Form. C | Glycerin - | (Full Form. - | (Full Form. - | Form. G | Form. H |
| | Lubricating | Trehalose - | (Full Form. - | Trehalose - | Glycerin - | Glycerin - | (Full Form. - | (Full Form. - |
| Ingredient (% w/v) | Eye Drops | Vitamins-AA) | Vitamins-AA) | Vitamins) | Trehalose-AA) | Trehalose) | AA) | Vitamins) |
| Glycerin | 0.750 | | x | | | | x | x |
| Trehalose dihydrate | 0.500 | | x | | | | x | x |
| Sodium hyaluronate | 0.240 | x | x | x | x | x | x | x |
| Sodium chloride | 0.300 | x | x | x | x | x | x | x |
| Potassium chloride | 0.140 | x | x | x | x | x | x | x |
| Magnesium ascorbyl | 0.100 | | | | x | x | x | |
| phosphate | | | | | | | | |
| Sodium | 0.060 | x | x | x | x | x | x | x |
| L-arginine | 0.050 | | | x | | x | | x |
| Citric acid anhydrous | 0.024 | x | x | x | x | x | x | x |
| L-Taurine | 0.020 | | | x | | x | | x |
| L-Leucine | 0.020 | | | x | | x | | x |
| Calcium chloride | 0.020 | x | x | x | x | x | x | x |
| Sodium bicarbonate | 0.005 | x | x | x | x | x | x | x |
| Cyanocobalamin (B₁₂) | 0.004 | | | | x | x | x | |
| Sodium hydroxide | 0.046 | x | x | x | x | x | x | x |
| Purified water | Q.S. to 100 | x | x | x | x | x | x | x |
| | % volume | | | | | | | |

*AA = Amino Acids

All products were tested as described in Table 8, below. Each test formulation was adjusted with NaCl to 400, 450, or 500 mOsm/kg and cells were incubated for 24 hours. Each exposure was performed in triplicate. Upon completion, the cell culture supernatant was collected and analyzed for release of cytokines IL-6, chemokines IL-8 and MCP-1, and cell metabolic activity was determined by the AlamarBlue™ assay, as described below.

TABLE 8
Experimental Design and Schedule Summary

| Day 1-3 | Day 4 treatment (24 hours) | |
|---|---|---|
| Initial Riken-HCEC | 75% DMEM/F12:25% HBSS | |
| Culture Conditions | or dry eye drop product | Day 5 Analysis |
| Seed Riken-HCEC | HBSS | Cells were washed 1 |
| 96 well plate and | Full Formulation | time with 0.2 ml |
| allow to grow a | Formulation B | DMEM and added 0.1 |
| minimum of 72 hours | Full Formulation without Glycerin and | ml of 10% alamarBlue |
| | without Trehalose | in DMEM for 3 hours |
| | Full Formulation without Amino Acids | and fluorescence read |
| | Full Formulation without Vitamins | with BioTek Synergy |
| | Full Formulation without Amino Acids | plate reader |
| | and without Vitamins | |
| | Full Formulation without Glycerin | |
| | without Trehalose without Vitamins | |
| | Full Formulation without Glycerin | |
| | without Trehalose without Amino Acids | |

Cytokine Analysis: The cell supernatants were assayed using a Millipore Magpix kit for the detection of cytokine IL-6 and chemokine IL-8 and MCP-1 and were quantitated using a Magpix kit generated standard curve to determine concentrations.

Metabolic Activity Analysis: Riken-HCEC lines were assessed for metabolic activity, using AlamarBlue™. In brief, cells were washed 1× with 0.2 ml/well Dulbecco's Modified Eagle's medium (DMEM). The AlamarBlue™ solution was diluted in DMEM at 10% v/v, pre-warmed in a water bath, and 0.1 ml solution per well was added. Each plate was incubated for 3 hours at 37° C., 5% CO₂, and 95% relative humidity. Each plate was read on a fluorescent microplate reader and results were assayed using relative fluorescence units (RFUs).

Cell viability was assessed by comparing RFUs for each Riken-HCEC test group to the HBSS control.

Results

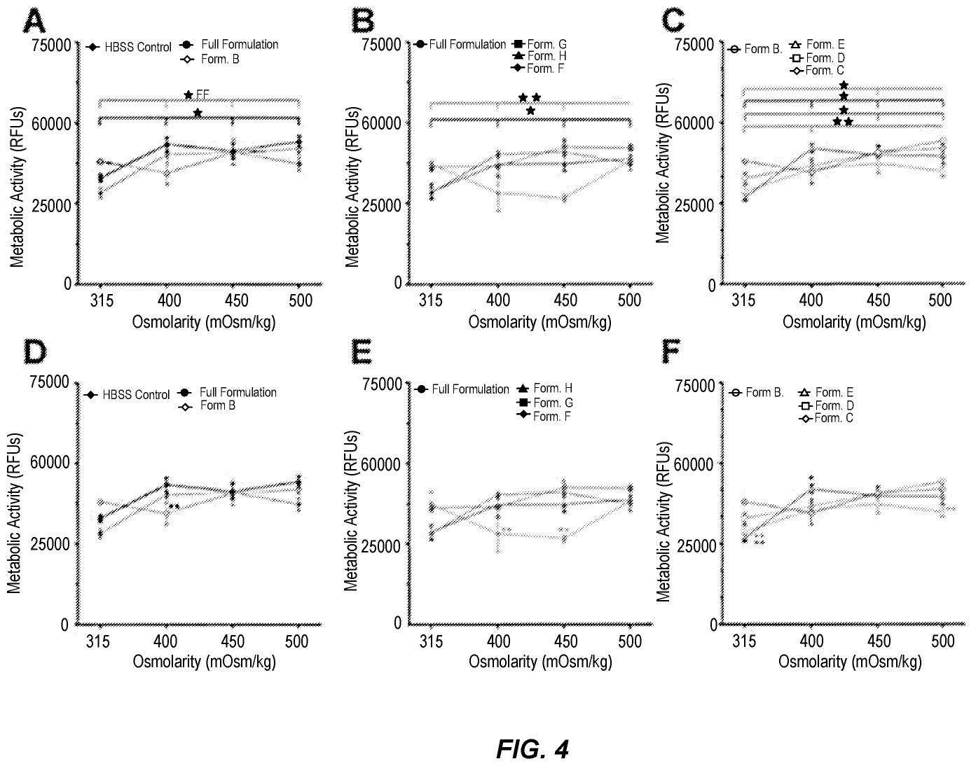

Metabolic Activity/Viability: Overall, there were significant increases in metabolic activity when the cells were exposed to hyperosmolarity. However, cells receiving Full Formulation without Glycerin and without Trehalose had significantly lower metabolic activity at 400 and 450 mOsm/kg versus the same solution at lower osmolarities (see FIG. 4).

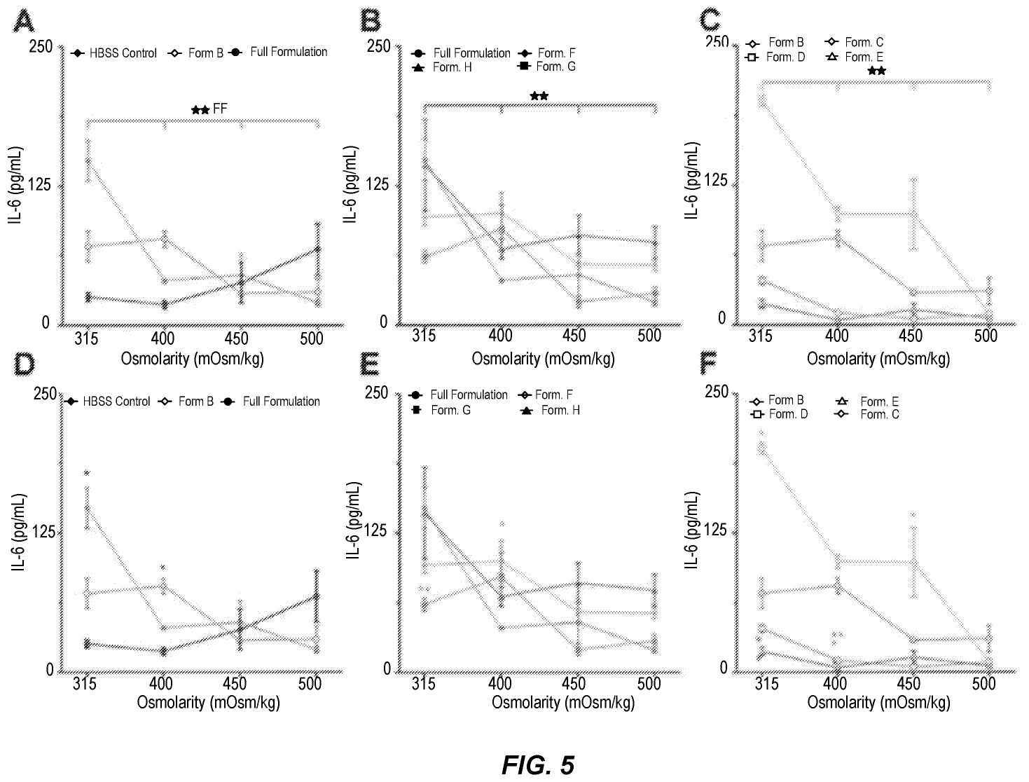

Cytokine Responses: Cells receiving the Full Formulation (FIGS. 5A and 5D) and Full Formulation without Amino Acids and without Vitamins (FIGS. 5C and 5F) had significant increases in IL-6 response at 315 mOsm/kg, but the response decreased with increasing osmolarity (see FIG. 5).

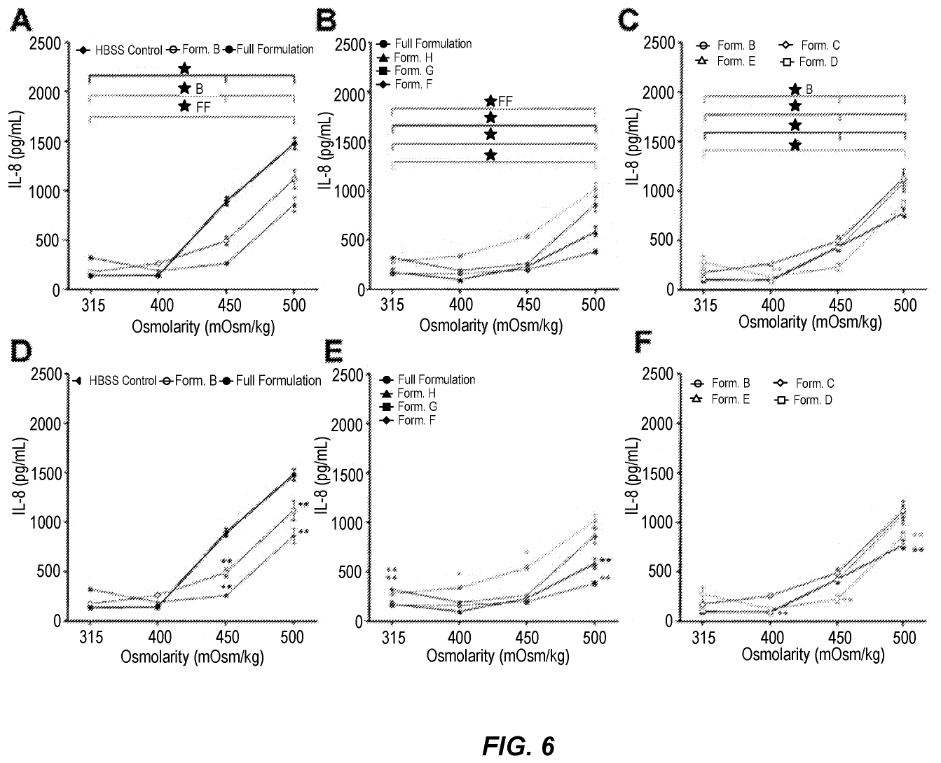

Chemokine Responses: Cells had significantly increased IL-8 responses with the HBSS control and Formulation B at 450 and 500 mOsm/kg and at 500 mOsm/kg as compared to the same formulations at 315 mOsm/kg control (FIG. 6A). When compared at the same osmolarity, the Full Formulation and the Formulation B had significantly decreased IL-8 responses at 450 and 500 mOsm/kg (FIG. 6D).

Cells receiving Full Formulation without Glycerin and without Trehalose had significantly higher IL-8 response at 450 and 500 mOsm/kg versus the 315 mOsm/kg control (FIG. 6B). Cells receiving Full Formulation without Vitamins or Full Formulation without Amino Acids had significantly higher IL-8 response at only 500 mOsm/kg using the same comparison (FIG. 6B). When compared at the same osmolarity, cells receiving Full Formulation without Glycerin and without Trehalose had significantly higher IL-8 response at 400 and 450 mOsm/kg (FIG. 6E). Cells receiving Full Formulation without Vitamins or Full Formulation without Amino Acids had significantly lower IL-8 responses at 315 mOsm/kg and 500 mOsm/kg.

Cells receiving Formulation B, Full Formulation without Glycerin without Trehalose and without Vitamins, or Full Formulation without Glycerin without Trehalose and without Amino Acids had significantly higher IL-8 response at 450 mOsm/kg and 500 mOsm/kg than at 315 mOsm/kg with the same formulation (FIG. 6C). The IL-8 response with cells receiving Full Formulation without Vitamins and without Amino Acids was significantly lower at 400 mOsm/kg and higher at 500 mOsm/kg versus the 315 mOsm/kg control. Cells receiving Full Formulation without Vitamins and without Amino Acids had significantly lower IL-8 response at 450 and 500 mOsm/kg versus the same base only osmolarity (FIG. 6F). Cells receiving Full Formulation without Glycerin without Trehalose and without Amino Acids had significantly lower IL-8 responses at 500 mOsm/kg. Cells exposed to Full Formulation without Glycerin without Trehalose and without Vitamins had significantly lower IL-8 response at 400 mOsm/kg response versus the control.

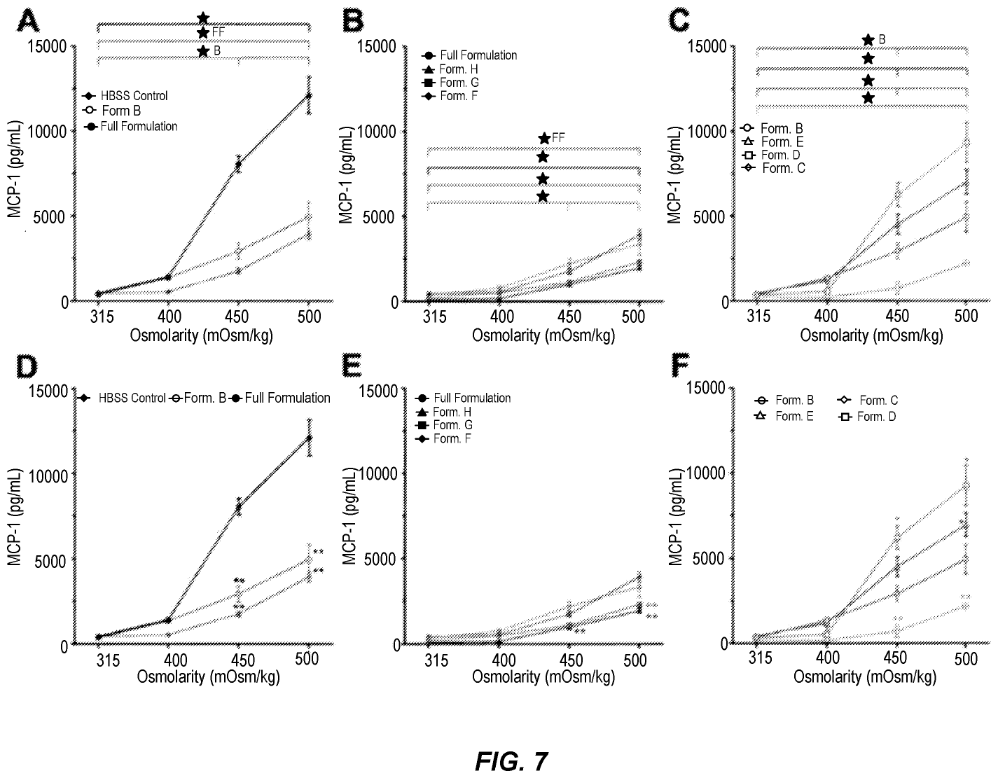

Cells receiving Full Formulation without Vitamins and without Amino Acids had significantly lower MCP-1 response at 450 mOsm/kg and 500 mOsm/kg versus the same base only osmolarity. (FIG. 7B.) Cells receiving Full Formulation without Glycerin without Trehalose and without Amino Acids had significantly higher MCP-1 response at 500 mOsm/kg than the base only control. (Id.) Cells exposed to Full Formulation without Glycerin without Trehalose and without Vitamins had significantly higher MCP-1 response at 450 and 500 mOsm/kg versus the base only control (see FIG. 7).

Summary: The data suggests that the Full Formulation without Glycerin and without Trehalose or the Full Formulation without Amino Acids and without Vitamins had the most significant effect on Riken-HCEC metabolic activity and IL-8 and MCP-1 responses. This indicates that glycerin and trehalose impart beneficial osmoprotectant qualities to the formulation.

What is claimed:

1. A formulation for hydrating eyes comprising at least one natural humectant, at least one amino acid, and at least one vitamin, wherein the formulation is preservative free.

2. The formulation according to claim 1, wherein the formulation is borate free.

3. The formulation according to claim 1 or 2, wherein the formulation is free of polyethylene glycol (PEG) and PEG-containing ingredients.

4. The formulation according to any one of claims 1-3, wherein the at least one natural humectant is chosen from glycerin, trehalose, sucrose, and sodium hyaluronate.

5. The formulation according to claim 4, wherein sodium hyaluronate is chosen from high molecular weight sodium hyaluronates.

6. The formulation according to claim 4 or 5, wherein the sodium hyaluronate has a molecular weight in the range of 700 kDa to 1500 kDa.

7. The formulation according to any one of claims 4-6, wherein sodium hyaluronate has a molecular weight of 800 kDa.

8. The formulation according to any one of claims 1-7, wherein the at least one amino acid is chosen from arginine, leucine, and taurine.

9. The formulation according to any one of claims 1-8, wherein the at least one vitamin is chosen from vitamin C and vitamin B₁₂.

10. The formulation according to claim 9, wherein vitamin C is chosen from sodium ascorbyl phosphate, magnesium ascorbyl phosphate, and combination thereof.

11. The formulation according to claim 9 or 10, wherein vitamin C is magnesium ascorbyl phosphate.

12. The formulation according to claim 9, wherein vitamin B₁₂ is chosen from cyanocobalamin, hydroxycobalamin, adenosyl cobalamin, and methyl cobalamin.

13. The formulation according to claim 9 or 12, wherein vitamin B₁₂ is cyanocobalamin.

14. The formulation according to any one of claims 1-13, wherein the formulation further comprises at least one natural electrolyte.

15. The formulation according to claim 14, wherein the at least one natural electrolyte is chosen from sodium chloride, potassium chloride, calcium chloride, and magnesium chloride.

16. The formulation according to any one of claims 1-15, wherein the formulation further comprises at least one natural buffer agent.

17. The formulation according to claim 16, wherein the at least one natural buffer agent is chosen from sodium citrate, citric acid, sodium bicarbonate, and amino acids.

18. The formulation according to any one of claims 1-17, wherein the pH of the formulation is in a range of 6 to 8.

19. The formulation according to any one of claims 1-18, wherein the pH of the formulation is 7.5.

20. The formulation according to any one of claims 1-19, wherein the osmolality of the formulation is 200 mOsm/kg to 300 mOsm/kg.

21. The formulation according to any one of claims 1-20, wherein the osmolality of the formulation is 275 mOsm/kg.

22. The formulation according to any one of claims 1-21, wherein the formulation is stable.

23. The formulation according to claim 1, wherein the formulation comprises glycerin, trehalose, sodium hyaluronate, sodium chloride, potassium chloride, magnesium ascorbyl phosphate, sodium citrate, L-arginine, citric acid, L-taurine, L-leucine, calcium chloride, sodium bicarbonate, magnesium chloride, cyanocobalamin, and sodium hydroxide.

24. The formulation according to claim 1, wherein the formulation comprises:
0.6% w/v to 0.8% w/v glycerin;
0.25% w/v to 0.5% w/v trehalose;
0.1% w/v to 0.3% w/v sodium hyaluronate;
0.2% w/v to 0.3% w/v sodium chloride;
0.01% w/v to 0.14% w/v potassium chloride;
0.01% w/v to 0.1% w/v magnesium ascorbyl phosphate;
0.02% w/v to 0.06% w/v sodium citrate;
0.02% w/v to 0.05% w/v L-arginine;
0.01% w/v to 0.0275% w/v citric acid;
0.01% w/v to 0.02% w/v L-taurine;
0.01% w/v to 0.02% w/v L-leucine;
0.01% w/v to 0.02% w/v calcium chloride;
0.010% w/v to 0.015% w/v sodium bicarbonate;
0.005% w/v to 0.011% w/v magnesium chloride;
0.001% w/v to 0.004% w/v cyanocobalamin; and
qs % w/v sodium hydroxide.

25. The formulation according to claim 1, wherein the formulation comprises:
0.75% w/v glycerin;
0.5% w/v trehalose;
0.24% w/v sodium hyaluronate;
0.3% w/v sodium chloride;
0.14% w/v potassium chloride;
0.1% w/v magnesium ascorbyl phosphate;
0.06% w/v sodium citrate;
0.05% w/v L-arginine;
0.0275% w/v citric acid;
0.02% w/v L-taurine;
0.02% w/v L-leucine;
0.02% w/v calcium chloride;
0.015% w/v sodium bicarbonate;
0.011% w/v magnesium chloride;
0.004% w/v cyanocobalamin; and
qs % w/v sodium hydroxide.
